(19) United States
(12) Patent Application Publication
(10) Pub. No.: US 2004/0122378 A1
(43) Pub. Date: Jun. 24, 2004

(54) Safety hypodermic syringe

(76) Inventor: Fu-Yu Hsu, Tayuan Hsiang (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/322,552

(22) Filed: Dec. 19, 2002

(51) Int. Cl. A61M 5/00

(52) U.S. Cl. 604/240; 604/110

(57) ABSTRACT

The present invention relates to a safety hypodermic syringe. The inner rim of the top end of the locking socket is provided with at least one corresponding downward guide member, which is formed with a high and low side at front and rear end, respectively. Moreover, an entrance and a locking protrusion are disposed at the high and low side, respectively. In addition, an unhindered inner wall is formed beneath the guide member. The hollow body of the needle unit has a flange at the bottom thereof which has a protrusion at both sides thereof, respectively. The protrusions are inserted into the entrances at the higher side of the guide members, moved along the bottom side of the guide member and turned at a preset angle to reach the locking protrusions at the lower side thereof in position, respectively. Meanwhile, the internal wall of the hollow body is tightly pressed against the front portion of the needle unit holder.

### BACKGROUND OF THE INVENTION

1. Field of the Invention

The present invention relates to a safety hypodermic syringe, and more particularly, to a syringe whose needle unit can be rapidly inserted through guide members with a higher and a lower side into needle locking socket and stably secured within the needle unit holder.

2. Description of the Prior Art

The needle part of conventional hypodermic syringes easily injures the nursing people or cleaners when and after the hypodermic syringes are discarded. Therefore, a safety syringe was developed in which the needle part together with a push-pull rod after injection is pulled back to cylinder body thereof. Meanwhile, the needle part is destroyed by a push force. This kind of syringe is disclosed in e.g. TW 356013, 463639, 414085, 384709, 359621, 492328, 467752, 475449, 480185, 497976, 430565, 471322, 332433, 394027, 447310, U.S. Pat. No. 5,385,557 etc.

A safety syringe, disclosed in TW 356013 (or U.S. Pat. No. 5,993,419) and shown in FIGS. 9(A) and 9(B), utilizes a locking seat 12 which is inserted at the front end of a cylinder body 11. The needle part 13 makes use of male thread 131 screwed into the locking seat 12. After injection, the pull hook 141 of the push-pull rod 14 together with the locking seat 12 is pulled back into the cylinder body 11. However, the use of the threaded joint between the needle part 13 and the locking seat 12 is time-wasting and impractical. Moreover, people don't know when they are exactly tightened so that it's possible that they are not tightened enough or the locking seat 12 is turned with the over-tightening, thereby leading to leakage of injection solution. Even, the locking seat 12 falls into the cylinder body 11.

Another safety syringe, disclosed in U.S. Pat. No. 5,215,533 and shown in FIGS. 10(A) and 10(B), includes a locking seat 12 and a needle part 13 both of which are so secured that the threaded front end 111 of the cylinder body 11 is screwed into the locking seat 12. Meanwhile, a spring claw 112 holds the locking seat 12 in place. However, this embodiment also has the above-mentioned drawbacks of screwing method. Thus, no further descriptions thereof are given hereinafter.

A further safety syringe, disclosed in TW 359621 (or U.S. Pat. No. 5,899,887) and shown in FIGS. 11(A), 11(B) and 11(C), has a needle part 15 which is vertically inserted from top to bottom into a locking seat 16 at the front end of the cylinder body 11. Thus, the needle part 15 is also pulled back when the pull hook 141 of the push-pull rod 14 pulls back the locking seat 16. However, when the needle part 15 is vertically engaged into front end 111 of the cylinder body 11, the locking seat 16 is directly compressed and, therefore, moved downward. Besides, when the needle part 15 is moved with the locking seat 16 downwardly, the top part thereof is formed with free space without places for positioning the needle part 15. Thus, the needle part 15 is easily shaken or falls, thereby affecting the injection process. Furthermore, the needle part 15 is easily loosened and can't be smoothly pulled back together with the locking seat 16.

Furthermore, a safety syringe, disclosed in U.S. Pat. No. 5,533,970 and shown in FIG. 12, includes a needle part 17 having two L-shaped slots 171, 172 in opposite directions for receiving protrusions 112, 142 on the cylinder body 11 and the push-pull rod 14. However, the needle part 17 has to be upwardly inserted through the bottom of the cylinder body 11. Thus, the assembly is impractical. Moreover, the needle part 17 and the push-pull rod 14 have to be turned at a certain angle for engagement or disengagement. This is also impractical for nursing people.

Yet another safety syringe, disclosed in TW 436639 and shown in FIGS . 13(A) und 13(B), describes a needle part 18 which is upwardly inserted through the bottom of the cylinder body 11. However, this also has the same disadvantage of impractical assembly as the last embodiment has.

Yet a further safety syringe, disclosed in TW 447310 and shown in FIGS. 14(A), 14(B) und 14(C), describes a needle part 191 and a locking seat 19 both of which are upwardly inserted through the bottom of the cylinder body 11. Thereafter, nodules 192 on the circumference of the locking seat 19 are hooked at the bottom of hooked grooves 113 so that they are connected in through holes. However, this also has the same disadvantage of impractical assembly as the last two embodiments have. Moreover, when the pull hook 141 of the push-pull rod 14 is engaged with the locking seat 19, the pull hook 141 has to be turned at a certain angle to disengage the nodules 192 from the hooked grooves 113 for pulling back. Thus, the use is impractical, too.

### SUMMARY OF THE INVENTION

It is a primary object of the present invention to provide a safety hypodermic syringe whose needle unit is installed from the top of cylinder body. Besides, guide members with a higher and a lower side allow a rapid screwing of needle unit into a needle locking socket. Meanwhile, it is stably secured with the needle unit holder and won't be loosened therefrom.

It is another object of the present invention to provide a safety hypodermic syringe which facilitates injection operation for nursing people. The assembly is not only rapid but also practical. And the nursing people won't be injured by the needle.

It is a further object of the present invention to provide a safety hypodermic syringe whose sealing effect is excellent after the needle unit is assembled. Thus, the solution leakage can be avoided.

### BRIEF DESCRIPTION OF THE DRAWINGS

The accomplishment of this and other objects of the invention will become apparent from the following description and its accompanying drawings of which:

FIG. 1 is a perspective exploded view of the main structure of the present invention;

FIG. 2 is a top view of a locking socket of the present invention;

FIG. 3(A) is an unfolded plan view of an applicable embodiment of the locking socket of the present invention;

FIG. 3(B) is an unfolded plan view of another applicable embodiment of the locking socket of the present invention;

FIG. 3(C) is an unfolded plan view of a further applicable embodiment of the locking socket of the present invention;

FIG. 4 is a sectional view of the needle unit and the locking socket in a disengagement state;

FIG. 5 is a sectional view of the needle unit and the locking socket in an engagement state;

FIG. 6 is a first sectional view of the present invention in an application state;

FIG. 7 is a second sectional view of the present invention in an application state;

FIG. 8 is a third sectional view of the present invention in an application state;

FIGS. 9(A) and 9(B) are schematic drawings of TW 356013;

FIGS. 10(A) and 10(B) are schematic drawings of U.S. Pat. No. 5,215,533;

FIGS. 11(A), 11(B) and 11(C) are schematic drawings of TW 359621;

FIG. 12 is schematic drawings of U.S. Pat. No. 5,533,970;

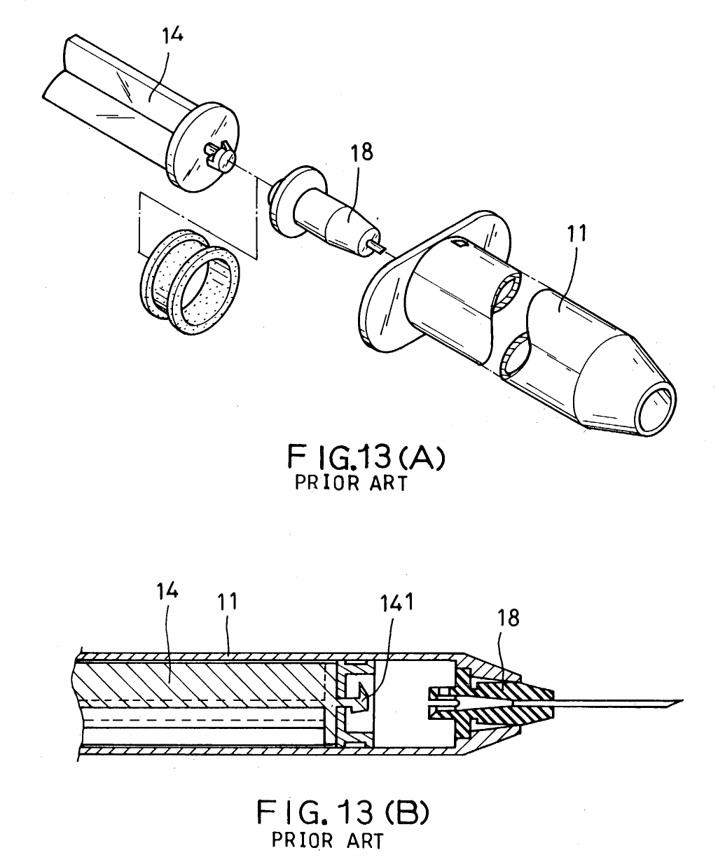

FIGS. 13(A) and 13(B) are schematic drawings of TW 463639; and

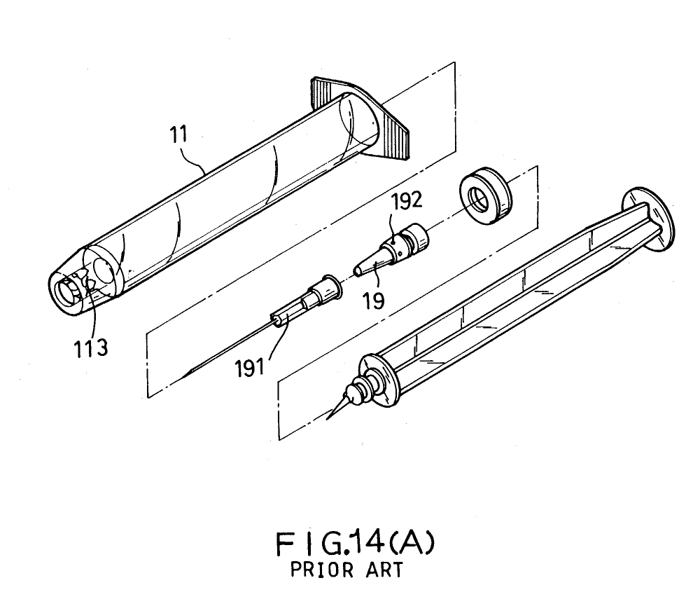

FIGS. 14(A), 14(B) and 14(C) are schematic drawings of TW 447310.

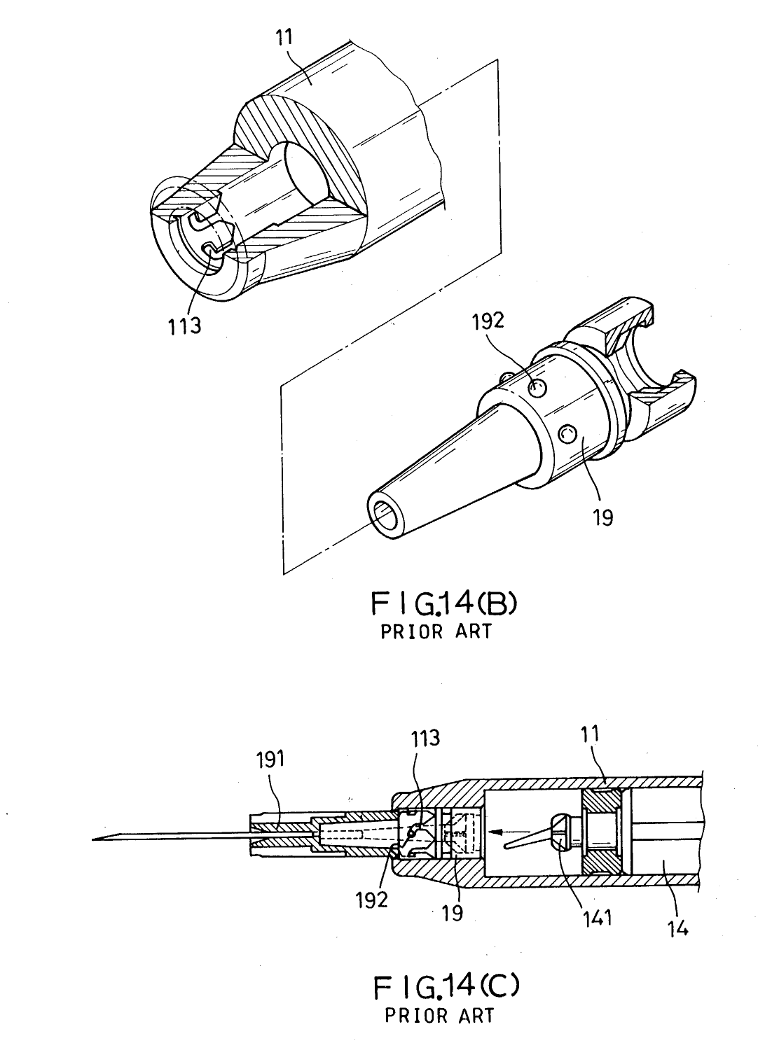

### DETAILED DESCRIPTION OF THE PREFERRED EMBODIMENT

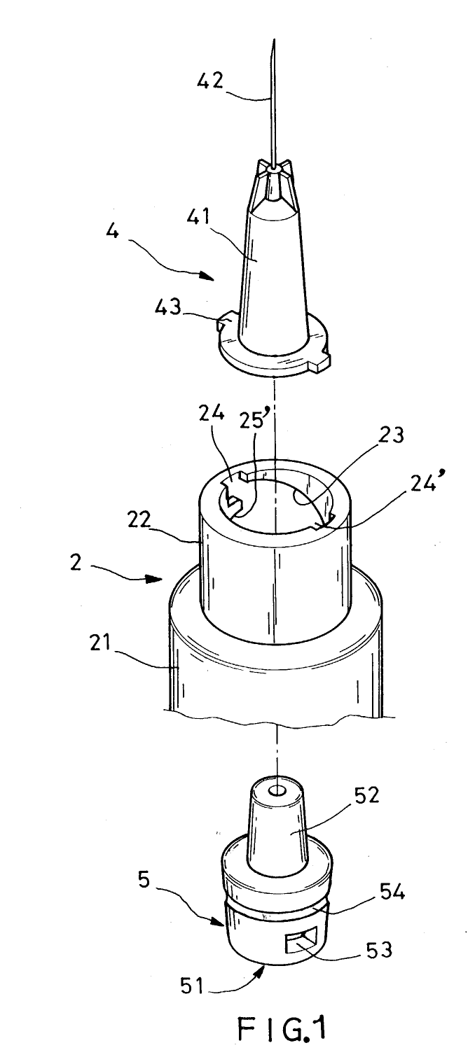

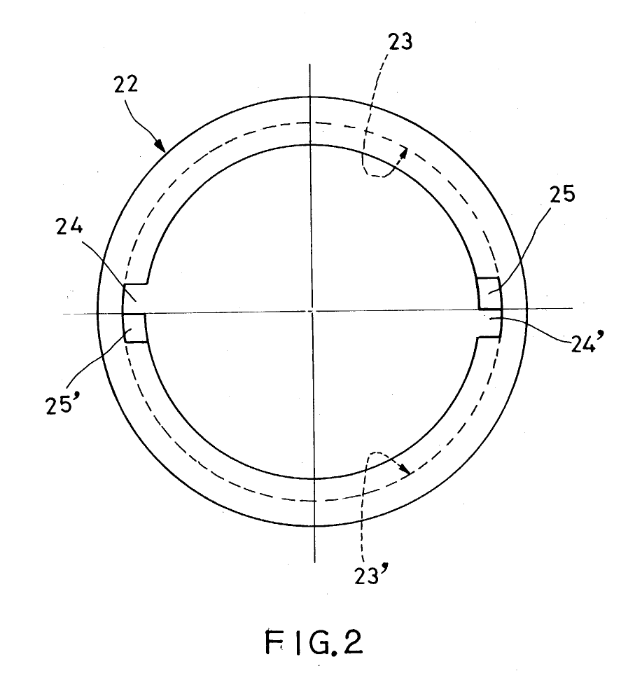

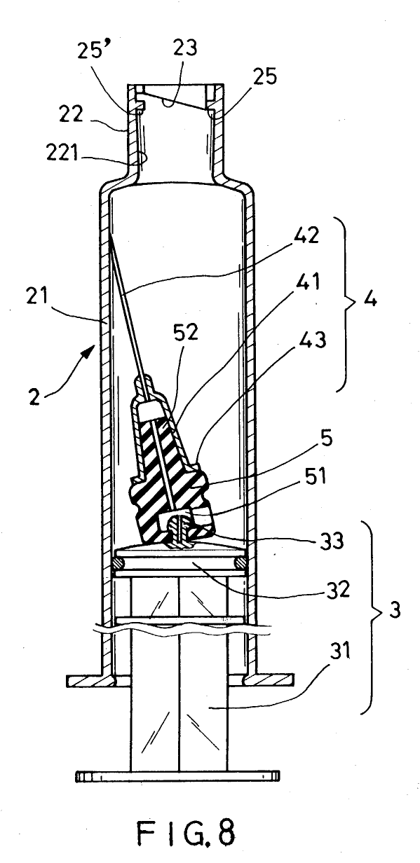

First of all, referring to FIGS. 1 and 2 together with FIG. 6, an improvement of the safety hypodermic syringe in accordance with the present invention is shown. The safety hypodermic syringe mainly includes a cylinder body 2, a push-pull rod 3, a push-pull rod 3, a needle unit 4 and a needle unit holder 5. The cylinder body 2 contains a hollow cylindrical barrel 21 and a locking socket 22 at front end of the hollow cylindrical barrel 21. The push-pull rod 3 consists of a plunger 31, a stopper 32 at the front end of the plunger 31 and a pulling hook 33 protruding at front end of the pulling hook 32. The needle unit 4 has a hollow body 41 and a needle 42 at front end of said hollow body 41. The needle unit holder 5 is inserted into the locking socket 22 from bottom to top. An engaging groove 51 corresponding to the pulling hook 33 is disposed at bottom of the needle unit holder 5 while a front portion 52 is secured with the hollow body 41 of the needle unit 4.

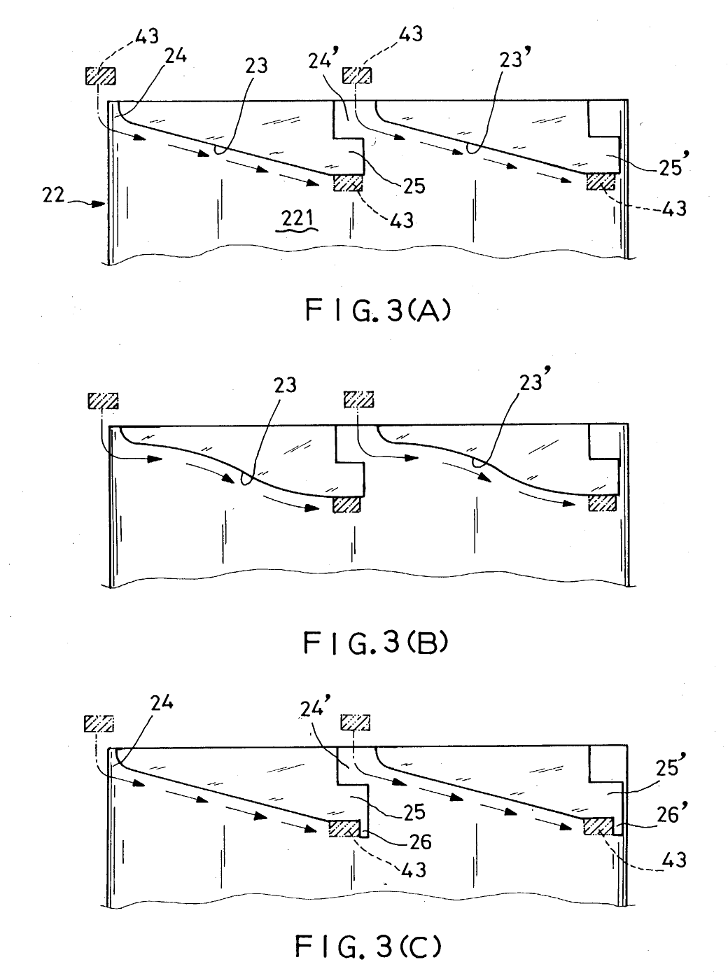

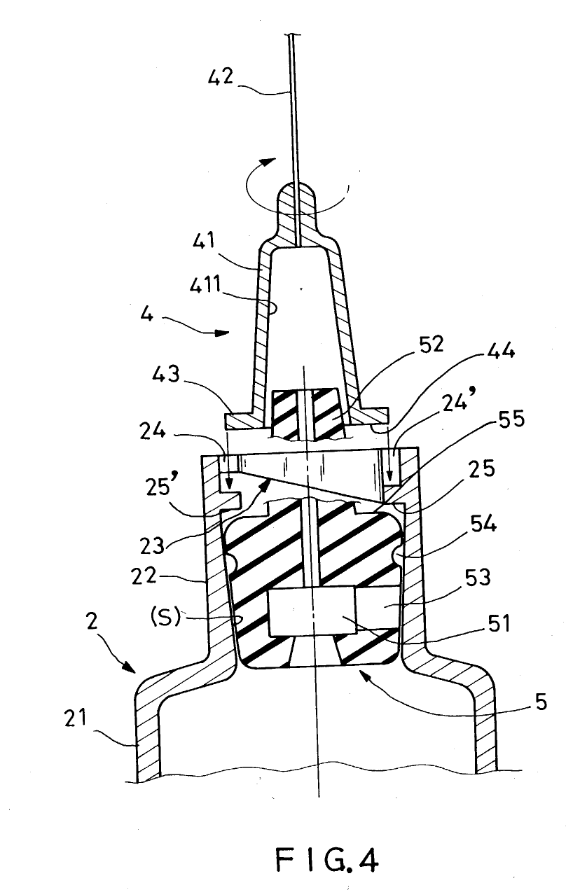

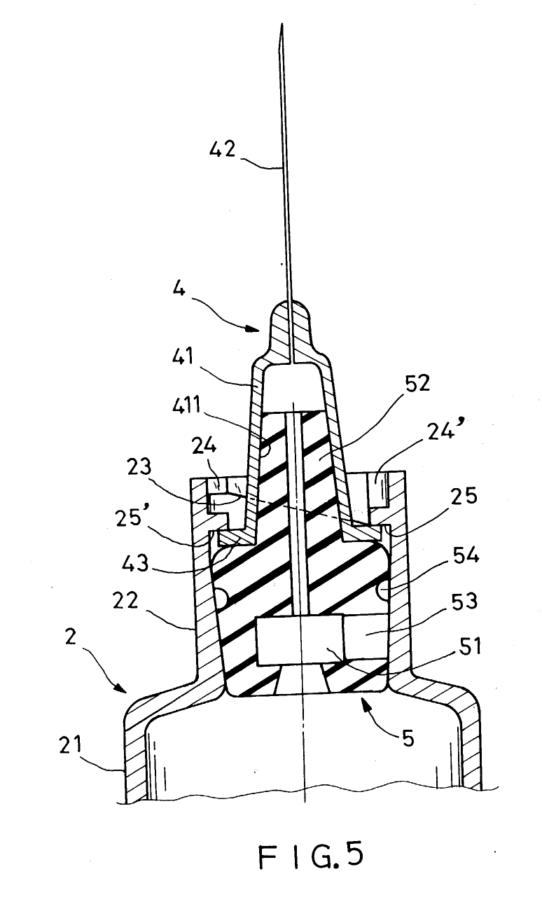

The inner rim of the top end of the locking socket 22 is provided with at least one corresponding downward guide member 23, 23′, which is formed with a high and low side at front and rear end, respectively. FIG. 3(A) shows an unfolding plan view thereof. Moreover, an entrance 24, 24′ and a locking protrusion 25, 25′ are disposed at the high and low side, respectively. In addition, an unhindered inner wall 221 is formed beneath the guide member 23, 23′.

The hollow body 41 of the needle unit 4 has a flange at the bottom thereof which has a protrusion 43 at both sides thereof, respectively. The protrusions 43 are inserted into the entrances 24, 24′ at the higher side of the guide members 23, 23′, moved along the bottom side of the guide member 23, 23′ and turned at a preset angle to reach the locking protrusions 25, 25′ at the lower side thereof in position, respectively. Meanwhile, the internal wall 411 of the hollow body 41 is tightly pressed against the front portion 52 of the needle unit holder 5.

FIG. 2 shows a top view of the locking socket of the present invention, and FIG. 3 shows an unfolded plan view of an applicable embodiment of the locking socket of the present invention. The outstanding feature of the present invention is the design of the guide members 23, 23′ with a higher and a lower side. The higher side thereof is located at the entrances 24, 24′ at the top rim of the locking socket 22 while the protrusions 43 at both sides of the hollow body 41 can be inserted right into the entrances 24, 24′. When the user turns the hollow body 41 at an angle within180°, the protrusions 43 are moved along the bottom rim of the guide members 23, 23′ downwards. Meanwhile, the the lower sides of the guide members 23, 23′ are constructed as horizontally extending support piece 25, 25′. When the protrusions 43 reach the locking protrusions 25, 25′, they will be suppressed by the locking protrusions 25, 25′. In addition to the aforementioned inclined structure, the guide members 23, 23′, as shown in FIG. 3(B), can be constructed in arched type. Furthermore, the locking protrusions 25, 25′, as shown in FIG. 3(C), can be provided with an end stop 26, 26′, respectively so that the protrusions 43 are able to exactly reach the expected position.

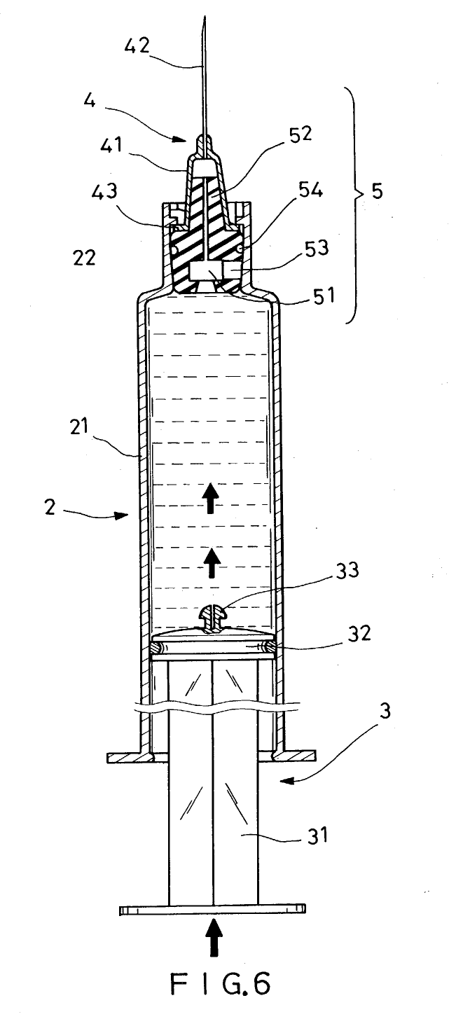

FIG. 4 shows a sectional view of the needle unit and the locking socket in a disengagement state. The needle unit holder 5 is pre-arranged within the locking socket 22. In order to ensure the connection therebetween, the inner wall 221 of the locking socket 22 is downwardly tapered while the external wall of the needle unit holder 5 is downwardly tapered, correspondingly. The end rims of the locking socket 22 and the needle unit holder 5 are rounded. Before the needle unit 4 is inserted into the locking socket 22, the needle unit holder 5 is located within the locking socket 22 in loose state so that a slight clearance (S) is existing. This design is used to avoid the leakage of injection solution. The feature and the function of this arrangement are described as follows.

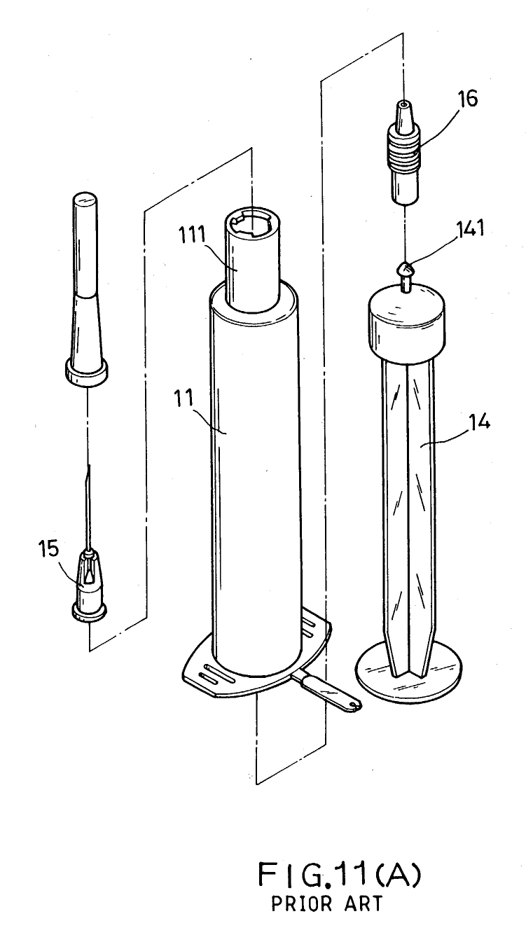

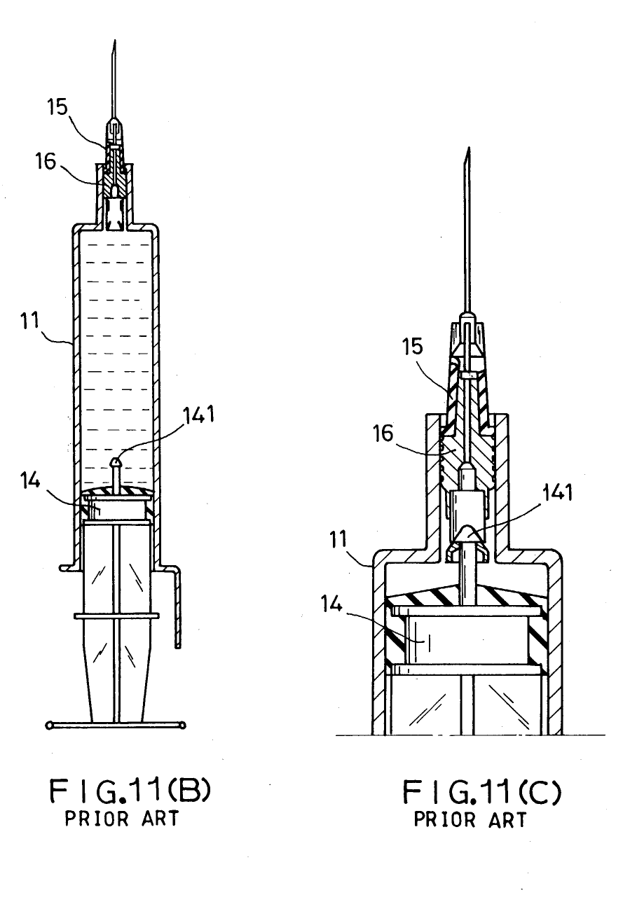

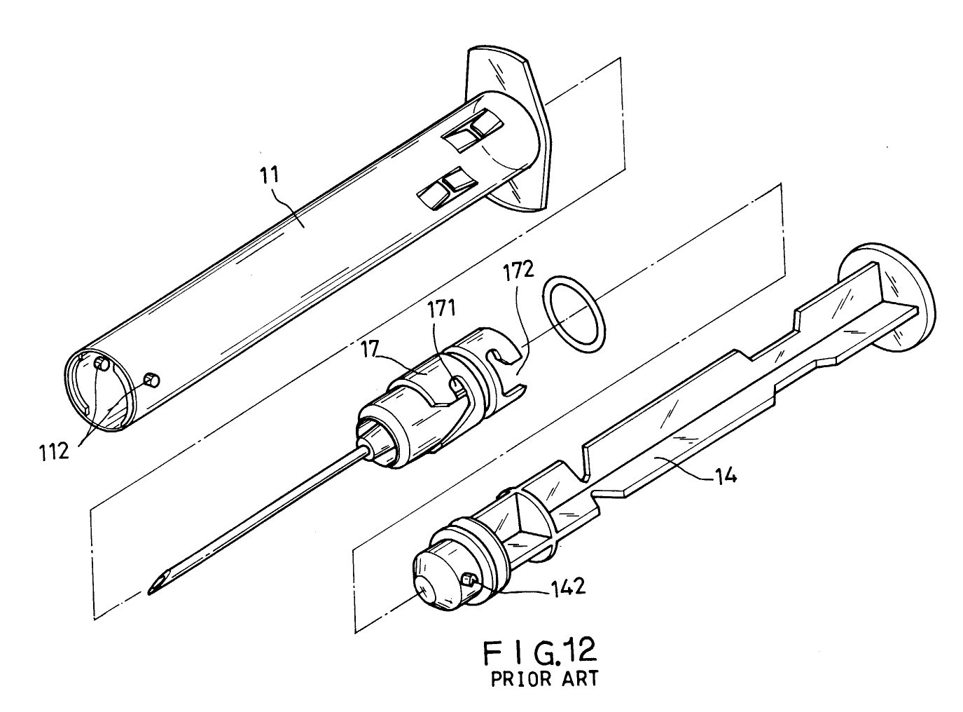

In order to prevent the needle unit 4 and the needle unit holder 5 from leakage, the present invention features that the needle unit 4 is inserted into the locking socket 22 only when the syringe has to be used. Accordingly, the structure of the needle unit 4 can't be too complicated; otherwise, the use is time-wasting and easily causes unnecessary mistake in emergency. For example, the bolted connection of the conventional syringe, as shown in FIGS. 9 and 10, has time-wasting drawback, while the pressurized connection of another conventional syringe, as shown in FIG. 11, has drawbacks of unstable positioning and solution leakage.

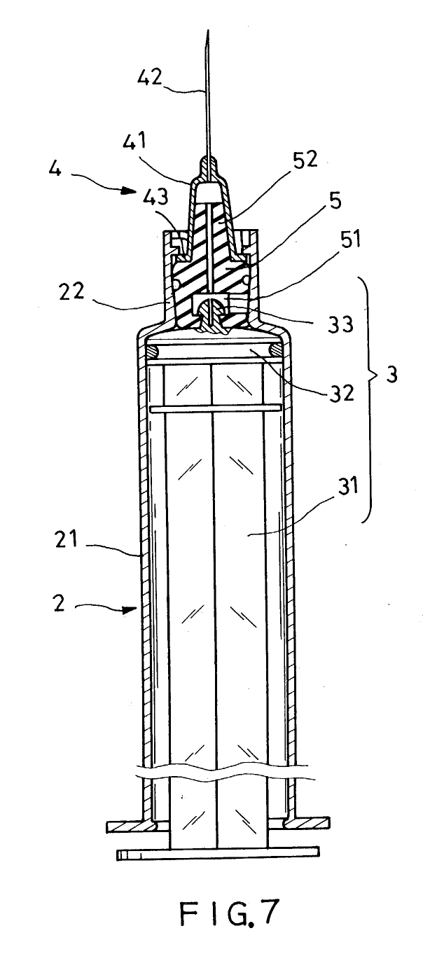

Accordingly, the guide member 23 disposed at the inner rim of the top of the locking socket 22 is used for rapidly mounting the needle unit 4. As shown in FIG. 4, when the needle unit 4 is inserted into the locking socket 22, the protrusions 43 at both sides thereof are in alignment with with entrances 24, 24′ of the guide members 23, 23′. Thereafter, the needle unit 4 is clockwise turned at an angle within 180° while the bottom side 44 will be first in pressing contact with the shoulder 55 of the needle unit holder 5, thereby compressing the needle unit holder 5 downward to be in tight contact with the locking socket 22. When the needle unit 4, as shown in FIG. 5, is reached in place, both protrusions 43 are situated right beneath the locking protrusions 25, 25′, thereby creating a squeezing effect on the needle unit holder 5. Meanwhile, the conic internal wall 411 of the hollow body 41 is in tight contact with the front portion 52 of the needle unit holder 5. Accordingly, the assembly is finished, and it only requires 1-2 seconds for mounting the needle unit 4 onto the locking socket 22. Thus, the present invention is easy to use and the users won't be injured by the needle 42.

Furthermore, as shown in FIG. 6, the needle unit holder 5 and the needle unit 4 of the present invention are tightly connected only in use. Unlike the conventional syringes whose needle unit holder 5 is pre-arranged within the locking socket 22, the present invention has no problems with solution leakage through slight clearance caused by oxidation, expansion or contraction of material.

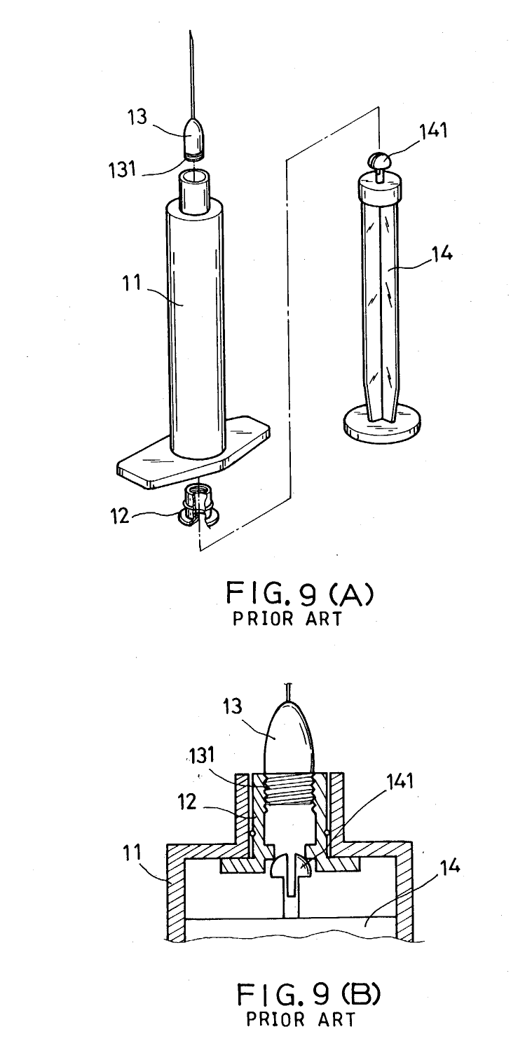

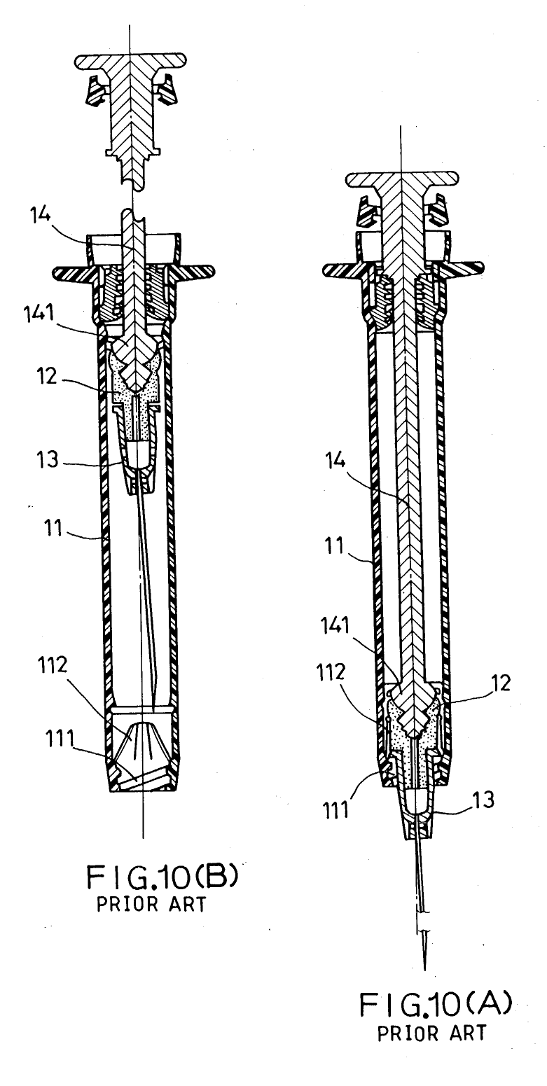

Then, referring to FIG. 7, when the injection is completed, the pulling hook 33 of the push-pull rod 3 is inserted into the engaging groove 51 so as to pull back the needle unit 4 and the needle unit holder 5. As shown in FIG. 8, the inner wall 221 beneath the needle unit 4 is unhindered except the locking protrusions 25, 25′ thereabove so that the needle unit 4 and the needle unit holder 5 can be smoothly pulled back within the cylinder body 2.

Moreover, the needle unit holder 5 can be provided with an annular groove 54 in which a sealing ring (not shown), if required, is placed. Furthermore, in order to facilitate the mold release after forming the engaging groove 51, a through hole 53 is disposed at one side of the engaging groove 51. These belong to prior art so that no further descriptions are given hereinafter.

Based on the above-mentioned configuration, the present invention provides a practical safety hypodermic syringe whose needle unit 4 is easy for insertion and a stable connection is ensured after insertion. Besides, the needle unit 4 won't shake in injection, and the sealing effect of the whole structure is excellent. Furthermore, there is no problem with the solution leakage.

Many changes and modifications in the above-described embodiments of the invention can, of course, be carried out without departing from the scope thereof. Accordingly, to promote the progress in science and the useful arts, the invention is disclosed and is intended to be limited only by the scope of the appended claims.

What is claimed is:

1. A safety hypodermic syringe comprising:
a cylinder body having a hollow cylindrical barrel and a locking socket at front end of said hollow cylindrical barrel;
a push-pull rod having a plunger, a stopper at the front end of the plunger and a pulling hook protruding at front end of said pulling hook;
a needle unit having a hollow body and a needle at front end of said hollow body; and
a needle unit holder inserted into said locking socket from bottom to top, an engaging groove corresponding to the pulling hook being disposed at bottom of said needle unit holder while a front portion is secured with said hollow body of said needle unit;
wherein the improvement of the present invention is characterized by:
the inner rim of the top end of said locking socket being provided with at least one corresponding downward guide member, which is formed with a high and low side at front and rear end, respectively, an entrance and a locking protrusion being disposed at the high and low side, respectively, an unhindered inner wall being formed beneath the guide member; and
said hollow body of said needle unit having a flange at the bottom thereof which has a protrusion at both sides thereof, respectively, said protrusions being inserted into said entrances at the higher side of said guide members, moved along the bottom side of said guide member and turned at a preset angle to reach said locking protrusions at the lower side thereof in position, respectively, said internal wall of said hollow body being tightly pressed against said front portion of said needle unit holder.

2. The safety hypodermic syringe as claimed in claim 1 wherein said locking protrusions of said guide members are horizontally extended.

3. The safety hypodermic syringe as claimed in claim 2 wherein said locking protrusions are provided with an end stop, respectively.

4. The safety hypodermic syringe as claimed in claim 1 wherein said guide members are constructed in inclined form.

5. The safety hypodermic syringe as claimed in claim 4 wherein said guide members are constructed in arched form.

6. The safety hypodermic syringe as claimed in claim 1 wherein said inner wall of said locking socket is downwardly tapered.

7. The safety hypodermic syringe as claimed in claim 1 wherein said outer wall of the needle unit holder 5 in contact with said inner wall of said locking socket is downwardly tapered
